(19) United States
(12) Patent Application Publication
(10) Pub. No.: US 2016/0325060 A1
(43) Pub. Date: Nov. 10, 2016

(54) BREATHING APPARATUS AND OPERATION METHOD THEREOF

(71) Applicant: LEAD DATA INC., Hukou Township (TW)

(72) Inventors: CHIH-YANG HSIAO, Zhubei City (TW); SHU-HUNG LIN, Zhubei City (TW)

(21) Appl. No.: 15/130,245

(22) Filed: Apr. 15, 2016

(30) Foreign Application Priority Data
May 8, 2015 (TW) 104114824

(51) Int. Cl. A61M 16/00

(52) U.S. Cl. CPC A61M 16/0057; A61M 16/0003; A61M 2016/0027; A61M 2016/0033; A61M 2230/40; A61M 2230/005

(57) ABSTRACT

A breathing apparatus includes a pressure regulating unit, a pressure detecting unit, a flow detecting unit and a processing unit. The pressure regulating unit has a coupling terminal. The pressure detecting unit is coupled to the coupling terminal and configured to detect an air pressure of the coupling terminal The flow detecting unit is coupled to the coupling terminal and configured to detect an air flow of the coupling terminal The processing unit is electrically coupled to the pressure regulating unit, the pressure detecting unit and the flow detecting unit. When the breathing apparatus is operated in a detecting mode, the processing unit is configured to configure the pressure regulating unit to modulate the air pressure to a detecting pressure within a detecting period according to a breath-in time parameter, a breathing frequency parameter and a pressure supporting parameter. An operation method of a breathing apparatus is also provided.

### TECHNICAL FIELD

The present invention relates to breathing apparatus and operation method thereof, and more particularly to breathing apparatus and operation method thereof suitable for central sleep apnea and obstructive sleep apnea.

### BACKGROUND

Sleep apnea is a sleep disorder apnea occurring when one is sleeping. In general, sleep apnea can be divided into central sleep apnea, obstructive sleep apnea and mixed sleep apnea containing the features of central sleep apnea and obstructive sleep apnea. Obstructive sleep apnea is caused by the soft tissues near the throat blocking the respiratory track and thereby resulting in breathing air not flowing smooth. Central sleep apnea is caused by brain stroke or traumatic and thereby resulting in brain cannot issue breathing commands. Patients with sleep apnea may suffer sleep problem, and which may lead to reduced focus and cognitive ability, mood swings, or even bad temper. To improve the sleep apnea situation, different breathing apparatuses are developed for different types of apnea situation. That is, if a patient has both of the central sleep apnea and the obstructive sleep apnea, he/she may needs two different types of breathing apparatus; therefore, the patient may need to pay more money and it is also quite inconvenient.

### SUMMARY

To overcome the aforementioned problems, the present invention provides a breathing apparatus, which includes a pressure regulating unit, a pressure detecting unit, a flow detecting unit and a processing unit. The pressure regulating unit has a coupling terminal. The pressure detecting unit is coupled to the coupling terminal and configured to detect an air pressure of the coupling terminal. The flow detecting unit is coupled to the coupling terminal and configured to detect an air flow of the coupling terminal. The processing unit is electrically coupled to the pressure regulating unit, the pressure detecting unit and the flow detecting unit. When the breathing apparatus is operated in a detecting mode, the processing unit is configured to configure the pressure regulating unit to modulate the air pressure to a detecting pressure within a detecting period according to a breath-in time parameter, a breathing frequency parameter and a pressure supporting parameter. The breath-in time parameter and the breathing frequency parameter are set based on an air resistance and the pressure supporting parameter is set based on lung softness.

The present invention further provides an operation method of a breathing apparatus. The breathing apparatus includes a pressure regulating unit, a pressure detecting unit, a flow detecting unit and a processing unit. The pressure regulating unit has a coupling terminal. The pressure detecting unit is coupled to the coupling terminal and configured to detect an air pressure of the coupling terminal. The flow detecting unit is coupled to the coupling terminal and configured to detect an air flow of the coupling terminal. The processing unit is electrically coupled to the pressure regulating unit. The pressure detecting unit and the flow detecting unit and configured to receive the air pressure and the air flow. The operation method of a breathing apparatus includes: when the pressure modulating unit is operated in a detecting mode, configuring the processing unit to configure the pressure modulating unit to modulate the air pressure to a detecting pressure within a detecting period according to a breath-in time parameter, a breathing frequency parameter and a pressure supporting parameter, wherein the breath-in time parameter and the breathing frequency parameter are set based on an air resistance and the pressure supporting parameter is set based on lung softness; and determining a status of the air flow.

In one embodiment, the aforementioned operation method of a breathing apparatus further includes: operating the pressure modulating unit in the detecting mode when the air flow is lower than a threshold.

In one embodiment, the aforementioned operation method of a breathing apparatus further includes: configuring the processing unit to determine whether the air flow has a change; if the air flow has a change, ending the detecting mode of the pressure modulating unit when the processing unit determines the air flow as a first status; and if the air flow has no change, ending the detecting mode of the pressure modulating unit and increasing the air pressure of the coupling terminal when the processing unit determines the air flow as a second status.

### BRIEF DESCRIPTION OF THE DRAWINGS

The present disclosure will become more readily apparent to those ordinarily skilled in the art after reviewing the following detailed description and accompanying drawings, in which:

FIG. 1 is a schematic structural diagram of a breathing apparatus in accordance with an embodiment of the present invention;

FIG. 2A is a plot showing an waveform of air flow versus time derived from a user having a central sleep apnea in sleep;

FIG. 2B is a plot showing a waveform of air pressure versus time of the coupling terminal derived from the breathing apparatus having an operation corresponding to FIG. 2A;

FIG. 3A is a plot showing an waveform of air flow versus time derived from a user having an obstructive sleep apnea;

FIG. 3B is a plot showing a waveform of air pressure versus time of the coupling terminal derived from the breathing apparatus having an operation corresponding to FIG. 3A; and

FIG. 4 is a flowchart of an operation method of a breathing apparatus in accordance with an embodiment of the present invention.

### DETAILED DESCRIPTION OF PREFERRED EMBODIMENTS

The present disclosure will now be described more specifically with reference to the following embodiments. It is to be noted that the following descriptions of preferred embodiments of this disclosure are presented herein for purpose of illustration and description only. It is not intended to be exhaustive or to be limited to the precise form disclosed.

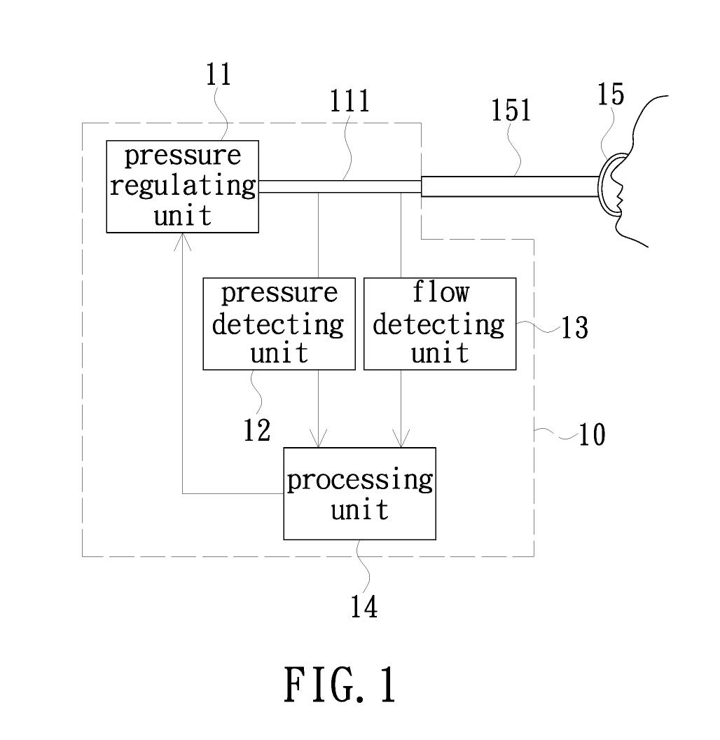

Refer to FIG. 1, which is a schematic structural diagram of a breathing apparatus in accordance with an embodiment of the present invention. As shown in FIG. 1, the breathing apparatus 10 of the present embodiment includes a pressure regulating unit 11, a pressure detecting unit 12, a flow detecting unit 13 and a processing unit 14. The pressure regulating unit 11 is electrically coupled to the processing unit 14 and has a coupling terminal 111. The pressure regulating unit 11 is configured to regulate an air pressure of the coupling terminal 111. The coupling terminal 111 is coupled to a breathing tube 151 which is coupled to a mask 15. While a user wears the mask 15 in sleep, the respiratory flow outputted from the user can be transmitted to the coupling terminal 111 through the breathing tube 151. The pressure detecting unit 12 is coupled to the coupling terminal 111 and electrically coupled to the processing unit 14. The pressure detecting unit 12 is configured to detect an air pressure of the coupling terminal 111. In one embodiment, the pressure detecting unit 12 is a mercury pressure meter. The flow detecting unit 13 is coupled to the coupling terminal 111 and electrically coupled to the processing unit 14. The flow detecting unit 13 is configured to detect an air flow of the coupling terminal 111. In one embodiment, the flow detecting unit 13 is a differential pressure flow meter or a volume flow meter. The processing unit 14 is electrically coupled to the pressure regulating unit 11, the pressure detecting unit 12 and the flow detecting unit 13. The processing unit 14 is configured to receive the aforementioned air flow and air pressure (e.g., signals corresponding to the aforementioned air flow and air pressure respectively detected from the flow detecting unit 13 and the pressure detecting unit 12) and determine whether the pressure regulating unit 11 is needed to be operated in a detecting mode according to whether the air flow of the coupling terminal 111 is lower than a threshold, such as: a minimum air flow.

In the present embodiment, the pressure regulating unit 11 is operated in the detecting mode when the air flow of the coupling terminal 111 is lower than the threshold. The processing unit 14 then configures the pressure regulating unit 11 to modulate the air pressure of the coupling terminal 111 to a detecting pressure within a detecting period according to a breath-in time parameter, a breathing frequency parameter and a pressure supporting parameter. In the present embodiment, the breath-in time parameter is set based on an air resistance of the user who is using the breathing apparatus 10; and the pressure supporting parameter is set based on lung softness. Specifically, the breath-in time parameter is related to the air resistance resulted by the user's trachea; the breathing frequency parameter is related to the user's breathing frequency; and the pressure supporting parameter is related to the softness of the user's lung. In the present embodiment, the aforementioned parameters are pre-installed in the breathing apparatus 10. In the present embodiment, specifically, the value of the detecting pressure is determined based on the breath-in time parameter and the pressure supporting parameter derived from the user; and the frequency of the detecting pressure is determined based on the breathing frequency parameter derived from the user. The processing unit 14 further determines whether the user is having a central sleep apnea or an obstructive sleep apnea in accordance with the air flow of the coupling terminal 111 in the detecting period. For example, when the detecting pressure of the coupling terminal 111 causes a change of the air flow due to that the throat of the user is not clogged, it is determined that the user has a central sleep apnea. Alternatively, when the detecting pressure does not cause a change of the air flow, it is determined that the user has an obstructive sleep apnea.

As described above, the value and frequency of the detecting pressure are determined based on the breath-in time parameter, the breathing frequency parameter and the pressure supporting parameter derived from the current breathing condition of a user; therefore, when it is determined that the user has a central sleep apnea and the user does not have his/her throat clogged, the detecting pressure is higher than the lung pressure of the user within the detecting period and consequentially air can naturally flow into the lung of the user. Once the lung is filled with air, the lung pressure of the user is then lower than the current air pressure due to the frequency of the detecting pressure and consequentially air can naturally flow out from the lung of the user. In other words, the breathing apparatus 10 of the present embodiment can enforce the user having sleep apnea to breath by mechanical mean and the user can breathe spontaneously and normally after the detecting period. Specifically, when it is determined that the user has an obstructive sleep apnea, the processing unit 14 will configure the pressure modulating unit 11 to further increase the air pressure so as to prevent the user from having his/her throat clogged.

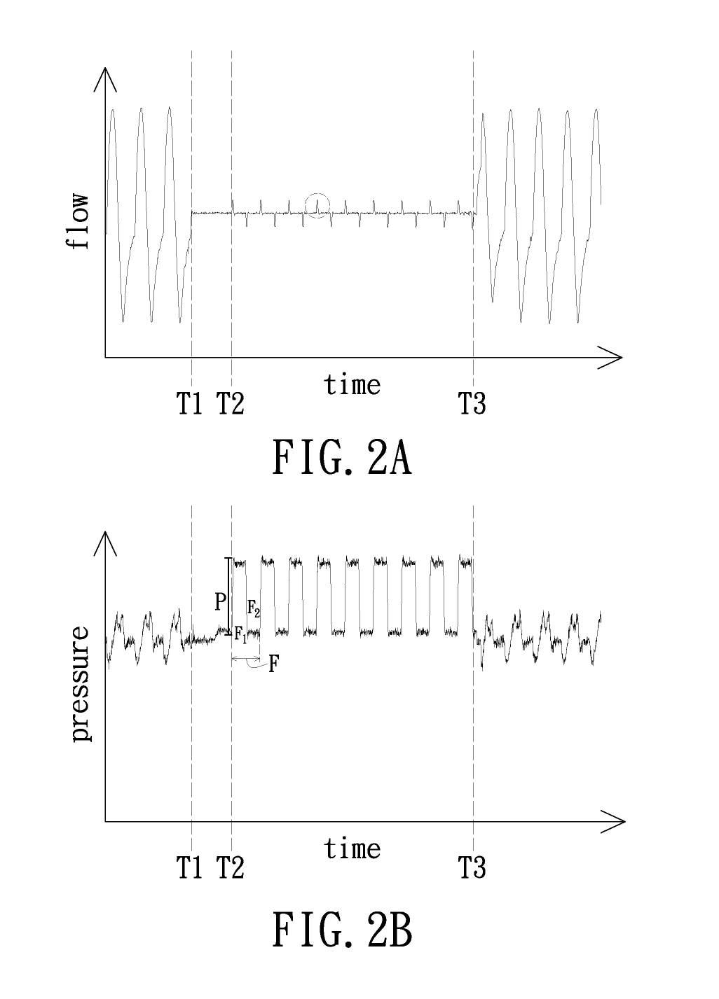

FIG. 2A is a plot showing a waveform of air flow versus time derived from a user having a central sleep apnea in sleep and FIG. 2B is a plot showing a waveform of air pressure versus time of the coupling terminal 111 derived from the breathing apparatus 10 having an operation corresponding to FIG. 2A. Hereunder the operation of the breathing apparatus 10 of the present embodiment will be described with a reference of FIG. 2A and FIG. 2B. First, as shown in FIG. 2A, the user has a relatively high air flow before the time point T1. Therefore, the processing unit 14 of the breathing apparatus 10 determines that the user can breathe spontaneously and normally before the time point T1. Then, as shown in FIG. 2A, the air flow of the user is relatively low and drops lower than a threshold at the time point T1. Therefore, the processing unit 14 configures the pressure regulating unit 11 to be operated in the detecting mode in response to a detection of the air flow of the user being lower than the threshold; and consequentially, the pressure regulating unit 11 modulate the air pressure of the coupling terminal 111 to the detecting pressure within the detecting period according to the pre-installed breath-in time parameter, breathing frequency parameter and pressure supporting parameter. That is, at the time point T2 in FIG. 2B, the air pressure of the coupling terminal 111 is modulated to the detecting pressure. In FIG. 2B, the value of the detecting pressure modulated based on the breath-in time parameter and the pressure supporting parameter is denoted by P; and the frequency of the detecting pressure modulated based on the breathing frequency parameter is denoted by F. Specifically, F₁ corresponds to the breath-in time of the user; and F₂ corresponds to the breath-out time of the user.

Once the air pressure of the coupling terminal 111 is modulated to the detecting pressure at the time point T2, the processing unit 14 then determines the type of the sleep apnea of the user according to the status of the air flow (e.g., the waveform of the air flow). In the present embodiment as illustrated in FIG. 2A, the air flow has a change (e.g., a pulse highlighted by a circle) caused by the detecting pressure after the time point T2. Therefore, the processing unit 14 determines that the user has a central sleep apnea; and consequentially, the pressure regulating unit 11 is back to an initial mode from the detecting mode at the end of the detecting period (that is, the time point T3); wherein the pressure regulating unit 11 in the initial mode is configured to stop modulating the air pressure of the coupling terminal 111. Because of the user is determined to have a central sleep apnea, the air pressure, modulated to the detecting pressure according to the breath-in time parameter, the breathing frequency parameter and the pressure supporting parameter which are set based on the current breathing condition of the user himself/herself within the detecting period, can help the user to breath normally and avoid the central sleep apnea. Thus, after the detecting period (that is, the time point T3), the breathing apparatus 10 can effectively solve the central sleep apnea of the user and the user can breathe spontaneously and normally.

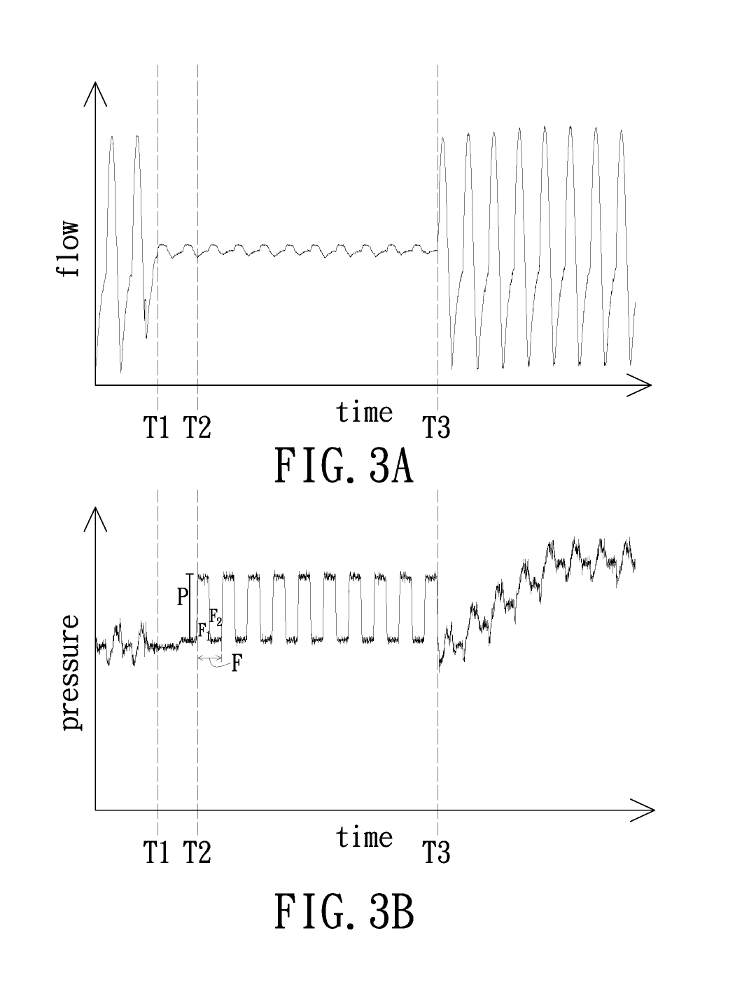

FIG. 3A is a plot showing a waveform of air flow versus time derived from a user having an obstructive sleep apnea in sleep and FIG. 3B is a plot showing a waveform of air pressure versus time of the coupling terminal 111 derived from the breathing apparatus 10 having an operation corresponding to FIG. 3A. Hereunder the operation of the breathing apparatus 10 of the present embodiment will be described with a reference of FIG. 3A and FIG. 3B. First, as shown in FIG. 3A, the user has a relatively high air flow before the time point T1. Therefore, the processing unit 14 of the breathing apparatus 10 determines that the user can breathe spontaneously and normally before the time point T1. Then, as shown in FIG. 3A, the air flow of the user is relatively low and drops lower than a threshold at the time point T1. Therefore, the processing unit 14 configures the pressure regulating unit 11 to be operated in the detecting mode in response to a detection of the air flow of the user being lower than the threshold; and consequentially, the pressure regulating unit 11 modulate the air pressure of the coupling terminal 111 to the detecting pressure within the detecting period according to the pre-installed breath-in time parameter, breathing frequency parameter and pressure supporting parameter. That is, at the time point T2 in FIG. 3B, the air pressure of the coupling terminal 111 is modulated to the detecting pressure. In FIG. 3B, the value of the detecting pressure modulated based on the breath-in time parameter and the pressure supporting parameter is denoted by P; and the frequency of the detecting pressure modulated based on the breathing frequency parameter is denoted by F. Specifically, F₁ corresponds to the breath-in time of the user; and F₂ corresponds to the breath-out time of the user.

Once the air pressure of the coupling terminal 111 is modulated to the detecting pressure at the time point T2, the processing unit 14 then determines the type of the sleep apnea of the user according to the status of the air flow (e.g., the waveform of the air flow). In the present embodiment as illustrated in FIG. 3A, the detecting pressure does not cause any change (e.g., a pulse) of the air flow after the time point T2. Therefore, the processing unit 14 determines that the user has an obstructive sleep apnea; and consequentially, the pressure regulating unit 11 further increase the air pressure of the coupling terminal 111 at the end of the detecting period (that is, the time point T3). Because of the user is determined to have an obstructive sleep apnea, the further-increased air pressure can help the user to breath normally and avoid the obstructive sleep apnea. Thus, after the detecting period (that is, the time point T3), the breathing apparatus 10 can effectively solve the obstructive sleep apnea of the user and the user can breathe spontaneously and normally.

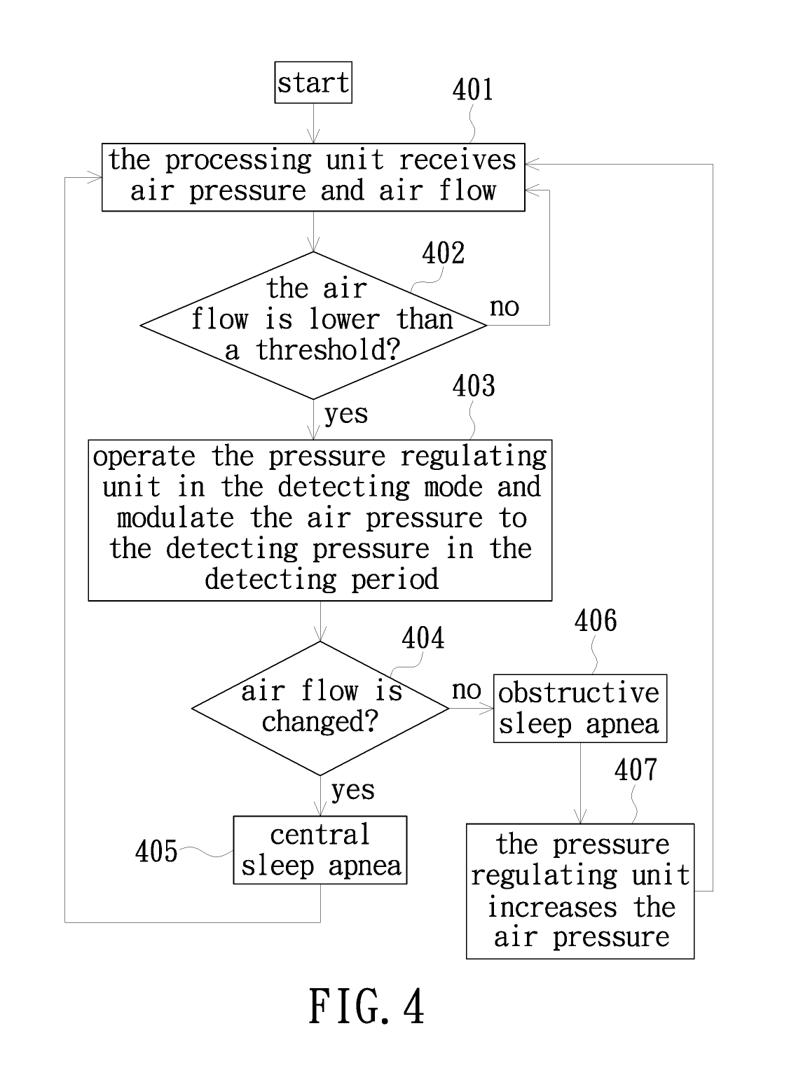

According to the above-mentioned contents, an operation method of a breathing apparatus is developed as illustrated in FIG. 4. As shown in FIG. 4, the processing unit 14 receives the air pressure and the air flow (e.g., signals corresponding to the aforementioned air flow and air pressure respectively detected from the flow detecting unit 13 and the pressure detecting unit 12) of the coupling terminal 111 (step 401). Then, the processing unit 14 determines whether the received air flow is lower than the threshold (step 402). If the received air flow is not lower than the threshold, the operation method goes back to the step 401 and the processing unit 14 continuously receives the air pressure and the air flow of the coupling terminal 111. Alternatively, if the received air flow is lower than the threshold, the processing unit 14 configures the pressure regulating unit 11 to be operated in the detecting mode and the pressure regulating unit 11 in the detecting mode modulate the air pressure of the coupling terminal 111 to the detecting pressure within the detecting period according to the breath-in time parameter, the breathing frequency parameter and the pressure supporting parameter (step 403). Then, the processing unit 14 determines whether the received air flow has a change caused by the detecting pressure (step 404). If the received air flow has a change, the processing unit 14 determines that the user has a central sleep apnea (step 405); and then the operation method goes back to the step 401. Alternatively, if the received air flow does not have a change, the processing unit 14 determines that the user has an obstructive sleep apnea (step 406). Then, the detecting period is over and the pressure regulating unit 11 further increases the air pressure of the coupling terminal 111(step 407); and then the operation method goes back to the step 401 for detecting a sleep apnea.

In summary, the breathing apparatus of the present invention can set the detecting pressure based on the breath-in time parameter, the breathing frequency parameter and the pressure supporting parameter derived from the current breathing condition of a user. Therefore, in the detecting period, the breathing apparatus of the present invention can enforce the user to breath, thereby effectively avoiding the central sleep apnea. Further, when it is determined that the user has an obstructive sleep apnea, the breathing apparatus of the present invention can further increase the air pressure so as to prevent the user from having his/her throat clogged. Therefore, the breathing apparatus of the present invention can help a user in sleep to breath either having central sleep apnea or obstructive sleep apnea.; and consequentially a user can have lower cost due to no need to use different breathing apparatuses according to different types of sleep apnea.

While the disclosure has been described in terms of what is presently considered to be the most practical and preferred embodiments, it is to be understood that the disclosure needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A breathing apparatus, comprising:
a pressure regulating unit, comprising a coupling terminal;
a pressure detecting unit, coupled to the coupling terminal and configured to detect an air pressure of the coupling terminal;
a flow detecting unit, coupled to the coupling terminal and configured to detect an air flow of the coupling terminal; and
a processing unit, electrically coupled to the pressure regulating unit, the pressure detecting unit and the flow detecting unit, wherein when the breathing apparatus is operated in a detecting mode, the processing unit is configured to configure the pressure regulating unit to modulate the air pressure to a detecting pressure within a detecting period according to a breath-in time parameter, a breathing frequency parameter and a pressure supporting parameter, wherein the breath-in time parameter and the breathing frequency parameter are set based on an air resistance and the pressure supporting parameter is set based on lung softness.

2. The breathing apparatus according to claim 1, wherein the breathing apparatus is operated in the detecting mode when the air flow is lower than a threshold.

3. The breathing apparatus according to claim 1, wherein the pressure detecting unit is a pressure meter.

4. The breathing apparatus according to claim 1, wherein the flow detecting unit is a differential pressure flow meter or a volume flow meter.

5. An operation method of a breathing apparatus, the breathing apparatus comprising a pressure regulating unit, a pressure detecting unit, a flow detecting unit and a processing unit, the pressure regulating unit comprising a coupling terminal, the pressure detecting unit being coupled to the coupling terminal and configured to detect an air pressure of the coupling terminal, the flow detecting unit being coupled to the coupling terminal and configured to detect an air flow of the coupling terminal, the processing unit being electrically coupled to the pressure regulating unit, the pressure detecting unit and the flow detecting unit and configured to receive the air pressure and the air flow, the operation method of a breathing apparatus comprising:
when the pressure modulating unit is operated in a detecting mode, configuring the processing unit to configure the pressure modulating unit to modulate the air pressure to a detecting pressure within a detecting period according to a breath-in time parameter, a breathing frequency parameter and a pressure supporting parameter, wherein the breath-in time parameter and the breathing frequency parameter are set based on an air resistance and the pressure supporting parameter is set based on lung softness; and determining a status of the air flow.

6. The operation method of a breathing apparatus according to claim 5, further comprising:
operating the pressure modulating unit in the detecting mode when the air flow is lower than a threshold.

7. The operation method of a breathing apparatus according to claim 6, further comprising:
configuring the processing unit to determine whether the air flow has a change;
if the air flow has a change, ending the detecting mode of the pressure modulating unit when the processing unit determines the air flow as a first status; and
if the air flow has no change, ending the detecting mode of the pressure modulating unit and increasing the air pressure of the coupling terminal when the processing unit determines the air flow as a second status.

8. The operation method of a breathing apparatus according to claim 7, wherein the first status indicates a central sleep apnea and the second status indicates an obstructive sleep apnea.
